(19) United States
(12) Patent Application Publication
(10) Pub. No.: US 2010/0316632 A1
(43) Pub. Date: Dec. 16, 2010

(54) MEANS AND METHODS FOR ENHANCING DIFFERENTIATION OF HAEMATOPOIETIC PROGENITOR CELLS

(76) Inventor: Stef Meers, Berchem (BE)

Correspondence Address: HOFFMANN & BARON, LLP, 6900 JERICHO TURNPIKE, SYOSSET, NY, 11791, US

(21) Appl. No.: 12/600,003

(22) PCT Filed: May 16, 2008
(86) PCT No.: PCT/NL2008/050293

§ 371 Date: Jan. 7, 2010

(30) Foreign Application Priority Data
May 16, 2007 (NL) PCT/NL2007/050228

(51) Int. Cl. A61K 39/395; C12N 5/071; C12N 5/078; C12N 5/0786; C12N 5/0783; C12N 5/0784; A61P 37/00

(52) U.S. Cl. 424/133.1; 435/325; 435/377; 424/172.1; 424/158.1

(57) ABSTRACT

The invention provides means and method for stimulating the production of differentiated haematopoietic cells in a culture comprising haematopoietic progenitor cells, lymphocytes (preferably T-cells) and monocytes/macrophages and/or dendritic cells. The methods involve among others culturing said cells or precursors thereof in the presence of a binding molecule specific for a co-stimulatory molecule expressed on said monocytes/macrophages and/or dendritic cells or said lymphocytes (preferably T-cells).

The myelodysplastic syndromes (MDS) are a heterogeneous group of haematopoietic diseases characterized by cytopenias, marrow dysplasia and an increased risk of development of leukemia.

The myelodysplastic syndromes were formerly referred to by many names including preleukaemia. The term preleukaemia is no longer used because it is misleading. Although a minority of patients with MDS develop acute leukaemia, most do not. When leukaemic transformation does occur, it is generally to acute myeloid leukaemia (AML). AML evolving from MDS is typically more difficult to treat than primary AML (cases arising in patients with no previous bone marrow disease).

The bone marrow in myelodysplastic syndrome is typically more active than normal and yet the numbers of blood cells in the circulation are reduced. This is because most of the cells being produced in the bone marrow are thought to be defective and/or destroyed before they leave the bone marrow to enter the blood stream. A reduction in numbers of all types of blood cell is called pancytopaenia. Another common feature of the myelodysplastic syndromes is abnormality in the appearance of the bone marrow and blood cells. These abnormalities (e.g. white cells lacking normal granules) are characteristic of the condition.

The myelodysplastic syndromes are difficult to treat because of the unusual combination of active marrow but inadequate blood cell production. The only treatment considered potentially curative is a donor stem cell transplant in younger and fitter patients. Unfortunately most patients are too old for this to be an option.

There is a degree of overlap between MDS and other marrow failure syndromes where an immune mediated pathogenesis has been proposed such as aplastic anaemia. It may sometimes be difficult to distinguish for instance between aplastic anaemia and a subtype of MDS in which the marrow is underactive.

MDS may be diagnosed at any age but is rare in childhood and uncommon in young adults. The median age at diagnosis is between 65 and 75 years, over 90% of patients are over 50 years at the time of diagnosis.

Men are more likely than women to develop MDS. This is most marked in the typical older patient group. Cases occurring in younger patients are more evenly distributed between men and women.

About twenty percent of cases arise in patients who have received either chemotherapy or radiotherapy as part of their treatment for another disease. This is known as secondary or therapy-related (t-MDS) treatment related MDS. This is more often the case with patients who develop MDS at a relatively young age.

There are 2 classification systems: FAB and WHO. In 1982, the French-American-British cooperative study group proposed guidelines for the classification of MDS. Morphologic dysplasia in the myeloid lineage and the number of bone marrow blasts divided patients into 5 distinct subclasses. In the 2001, the FAB-classification was revised and updated in the World Health Organization Classification of MDS. This classification was not only based on morphology, but included clinical, genetic, immunophenotypic and biologic findings to define MDS entities.

There are five types of myelodysplastic syndrome in the FAB system as it is currently used.

These are: refractory anaemia, refractory anaemia with ring sideroblasts, refractory anaemia with excess blasts, refractory anaemia with excess blasts in transformation and chronic myelomonocytic leukaemia

### Refractory Anaemia (RA)

The marrow cells that produce red cells appear abnormal. The white cell and platelet producing cells may also appear abnormal but the proportion of primitive cells (blast cells) is not significantly increased. A clinical feature of the disease is anaemia, which is usually mild to moderate but can be severe; often the red cells have a larger average size (mean cell volume or MCV) than normal, this is called macrocytosis. The numbers of white cells and/or platelets may be lower than normal.

RA accounts for about 30-45% of cases. About 10% of cases of RA will transform to acute leukaemia. Some patients with RA survive well in excess of five or even ten years, but the average survival ranges from two to five years.

Refractory Anaemia with Ring Sideroblasts (RARS)

The same changes are seen as in RA but there are additional abnormalities in the red cell population. The red cell precursors are unable to use iron normally and instead the iron is deposited in characteristic rings in the red cell precursors. These cells are called ring sideroblasts. If there are more than 15% ring sideroblasts in the bone marrow the disease is classified as RARS. While anaemia is again a common clinical problem, the numbers of white cells and/or platelets may also be lower than normal. The overall survival is the same as in RA but transformation to acute leukaemia is lower at about 8% of cases. This form of MDS makes up approximately 15% of cases.

Refractory Anaemia with Excess Blasts (RAEB)

In this form there is an increase in precursor blood cells (called blasts) in the marrow. Normal bone marrow contains up to about 5% blast cells.

Patients with RAEB have between 5-20% blast cells in their bone marrow. Patients with this form are more likely to have reduced numbers of platelets and/or white cells as well as red cells in their blood. This form accounts for about 15% of cases and has a median survival of about a year.

About 40% of patients with RAEB will go on to develop acute leukaemia.

The following subtypes are currently included in the FAB scheme but a new proposed classification of cancers of the blood and bone marrow produced by the World Health Organization has suggested that they be moved to other categories.

Refractory Anaemia with Excess Blasts in Transformation (RAEB-t)

The findings in these patients are similar to those in RAEB but with a higher proportion of blasts (20-30%) in the marrow. This form accounts for about 5-15% of cases. It has recently been proposed that these patients should now be classified as having acute myeloid leukaemia. The rate of conversion to overt leukaemia (over 30% blasts in the marrow) is high (between 60-75%) and the treatment is similar to that used for acute myeloid leukaemia.

The median survival is six months or less but chemotherapy, with or without stem cell transplantation, produces prolonged survival in some cases.

### Chronic Myelomoncytic Leukaemia (CMML)

In CMML the red cell precursors usually appear abnormal. The defining feature of CMML is that the number of one type of white cells (monocytes) in the blood is increased to more than 1×10⁹/litre. The marrow may or may not contain an increased proportion of blast cells. There may be anaemia and/or low platelets.

CMML is considered to be a form of myelodysplastic syndrome because the bone marrow shows features similar to those seen in other forms of the disease, but it also shows features of the related diseases known as the myeloproliferative disorders. The new WHO classification moves CMML into a separate category called the Myelodysplastic/Myeloproliferative Disorders.

CMML accounts for approximately 15% of myelodysplastic syndromes. Transformation of CMML to acute leukaemia happens in a similar way to other forms of myelodysplastic syndrome. Median survival is of the order of 12-18 months. Between 15-30% of patients progress to acute leukaemia.

Although most patients have no obvious cause for their disease there are several well established risk factors. Exposure to high levels of certain chemicals, particularly benzene, and to high levels of ionising radiation are both considered probable causes of MDS. An increased incidence of MDS has been reported among smokers and ex-smokers and is probably associated with specific chemicals present in tobacco smoke.

Not all patients will receive active treatment straight away because in most cases there is no evidence that early treatment influences overall survival duration. Patients who are not starting treatment will have regular check-ups. This is often referred to as watch-and-wait.

For the majority of patients the choice of treatment will be based on the International Prognostic Scoring System (IPSS) risk category, their age and their general fitness. The IPSS includes information on disease stage, the number of cell types affected and cytogenetics (the chromosome changes usually seen in MDS). These have all been found to be relevant in predicting the outcome (prognosis) and in selecting the optimal treatment. Four risk categories are recognized within the IPSS. These are low-risk, intermediate-1 and intermediate-2 risk and high-risk.

Growth factors are natural substances produced in the body which control the production and maturation of blood cells and which may be used in treatment. The major growth factors used in treatment of the myelodysplastic syndromes are erythropoietin (EPO), which stimulates production of red cells and factors called G-CSF/GM-CSF, which stimulate production of granulocytes (white cells). Unfortunately, erythropoietin appears to benefit only about 15% to 20% of patients with MDS and these are mainly patients with refractory anemia who are not dependent on blood transfusions. About 40% of MDS patients treated with a combination of EPO plus G-CSF will show improvement of their anemia. G-CSF alone may be used for short-term treatment during severe infection episodes that do not respond to conventional therapy.

### Experimental Therapies

There are several treatment approaches which are currently being tested in comparative studies (clinical trials) to determine their potential role in the treatment of MDS. These include the use of natural biochemicals called cytokines to try to induce MDS cells to undergo normal maturation.

The cytokines, which have been studied to date include growth factors, interleukins and interferons. It is not anticipated that cytokines will extend survival or induce remission, rather it is hoped that they will improve quality of life for MDS patients and reduce the need for transfusions and antibiotic treatment. As pancytopenia is the leading cause of morbidity and mortality in MDS (only a minority will transform into AML), improving cytopenias will increase patients' well-being.

Other promising reagents under current investigation are, amongst others, hypomethylating agents, signal pathways modulators, thalidomide and its analogue lenalidomide. The methyl-transferase inhibitors (5-azacytidine and 5-aza-2′-deoxycytidine are currently the only drugs that have FDA approval to treat MDS).

Thalidomide is a potent immune-modulating agent with a broad spectrum of immunologic effects and has been used in MDS patients as single-agent therapy (Musto P. et al., Leuk. Res. 2004(28)325-332; Raza A. et al., Blood 2001(98)958-965; Strupp C. et al., Leukemia 2002(16)1-6) or in combination with other agents (Steurer M. et al., Br. J. Haematol. 2003(121)101-103; Cortes J. et al., Cancer 2003(97)1234-1241; Raza A. et al., Leuk. Res. 2004(28)791-803; Candoni A. et al., Ann Hematol. 2005(84)479-481). Inhibition of TNF-α production is believed to be the primordial mechanism by which thalidomide acts in MDS.

### Immune Mediated Pathogenesis of MDS

The pathogenesis of BM failure in MDS is complex and is thought to be related to a delicate interplay between intrinsic defects in the haematopoietic progenitor cells and the BM microenvironment in which these progenitor cells reside (ref). There is increasing evidence that immune mechanisms play an important role in the pathogenesis of pancytopenia. This evidence comes from in vitro experiments that autologous T lymphocytes suppress MDS progenitor growth and reports of the presence of oligoclonal T cell expansions in selected patients, suggestive of an antigen-driven pathophysiology. An indication for a role of the immune system in the process of BM failure comes from the observation that up to 30% of low-risk MDS patients respond to immunosuppressive agents such as cyclosporine A (CSA) and/or anti-thymocyte globulin (ATG).

Most work has focused on the role of the lymphocytes in this immune process. In the present invention we show that CD40 expressing cells and in particular monocytes are part of the immune reaction that is going on in the BM of patients suffering from immune cell related bone marrow failure and that these CD40 expressing cells act in concert with lymphocytes. It was shown that CD40 stimulation of MDS monocytes leads to significantly increased TNF-alpha production. We further show that monocytes expression of co-stimulatory molecules (CD40, CD80 and CD86) is increased. Also the natural ligand of CD40, CD154 is significantly more expressed by T helper cells. We further show that molecules that specifically interact with one or more of these co-stimulatory molecules, in cultures of bone marrow cells from these individuals, result in higher numbers of produced progeny cells than comparable cultures without the binding molecule(s).

The invention in one embodiment provides a method for culturing a collection of cells comprising haematopoietic progenitor cells, CD40 receptor ligand expressing cells, preferably lymphocytes and CD40 receptor expressing cells, preferably monocytes/macrophages and/or dendritic cells comprising culturing said cells or precursors thereof in the presence of a binding molecule specific for a co-stimulatory molecule expressed on said CD40 receptor ligand expressing cells, preferably lymphocytes or CD40 receptor expressing cells, preferably monocytes/macrophages and/or dendritic cells. The invention further provides a method for stimulating the production of differentiated haematopoietic cells in a culture comprising haematopoietic progenitor cells, CD40 receptor ligand expressing cells, preferably lymphocytes and CD40 receptor expressing cells, preferably monocytes/macrophages and/or dendritic cells comprising culturing said cells or precursors thereof in the presence of a binding molecule specific for a co-stimulatory molecule expressed on said CD40 receptor ligand expressing cells, preferably lymphocytes and CD40 receptor expressing cells, preferably monocytes/macrophages and/or dendritic cells. Such cultures produce more differentiated cells than cultures without such binding molecules. Various types of cultures can be used. Cultures typically involve incubating the cells with culture medium. The phenomenon can be detected in “normal” cultures and in cultures on so-called feeder layers, such as is typically done in so-called long term bone marrow cultures. The haematopoietic progenitors may be present upon the start of the culture or produced from more primitive progenitor/stem cells that were introduced into the culture. As defined herein (haematopoietic) stem cells are cells that have the potential to give rise to at least any type of haematopoietic cell of the adult haematopoietic system. A haematopoietic stem cell or progenitor cell is derived from the blood system. Other types of stem cells such as embryonal stem cells are not included in the definition. Haematopoietic stem cells give rise to normal blood components including red cells, white cells and platelets. Stem cells are normally located in the bone marrow and in the blood and can be harvested for a transplant. Haematopoietic stem cells have extensive self renewal capacity. The stem cells differentiate into the mature end cells in various steps, through a cascade of more differentiated progenitor cells. These more differentiated progenitor cells have more limited self renewal capacity.

A method of the invention is particularly useful when at least either monocytes or lymphocytes are derived from an individual suffering from or at risk of suffering from immune cell mediated bone marrow failure. It is thought that the mentioned cells from these individuals are activated, or at least comprise a higher fraction of activated cells and that as a result the production of differentiated cells from more primitive progenitor cells is reduced. This at least in part explains the observed reduction of differentiated cells in the blood of these individuals. In a preferred embodiment said monocytes/macrophages and/or dendritic cells are derived from an individual suffering from or at risk of suffering from immune cell mediated bone marrow failure.

Non-limiting examples of immune cell mediated bone marrow failure diseases are for instance graft versus host disease (GVHD) and vice versa, Host versus graft (HVG) disease (HVGD). In a preferred embodiment said immune cell related bone marrow failure disease is a clonal bone marrow disorder. Preferred examples of such clonal disorders are the myelodysplastic syndromes (MDS), aplastic anemia, paroxysmal nocturnal hemoglobinuria (PNH), pure red cell aplasia (PRCA), myelofibrosis or large granular lymphocytic leukemia (LGL). Without being bound by theory it is thought that the immune mediated failure results in more active genesis of progenitor and differentiated cells. This more active genesis ultimately results in selection and thus clonality.

The present invention further provides a method for the treatment of cytopenia and/or bone marrow failure in an individual comprising providing said individual with a binding molecule specific for a co-stimulatory molecule expressed on monocytes/macrophages and/or dendritic cells or lymphocytes. Further provided is a method for the treatment of cytopenia and/or bone marrow failure in an individual comprising providing said individual with a binding molecule for reducing and/or inhibiting immune activation in said individual. The term cytopenia is herein defined as a reduction in the number of cells or a reduction in one type of cell, circulating in the blood. There are several types of cytopenia; low red blood cell count: anemia; low white blood cell count: leukopenia or neutropenia (because neutrophils make up at least half of all white cells, they are almost always low in leukopenia); low platelet count: thrombocytopenia; low red blood cell, white blood cell, and platelet counts: pancytopenia. The present invention is particularly suited for the treatment of the cytopenias' of the myeloid lineage.

In a method of the invention the use of a binding molecule of the invention is combined with a further medicament for the treatment of MDS.

In a preferred embodiment said further medicament comprises an immune-modulatory agent, chemotherapy or an antibody. The use of a binding molecule of the invention in such combination treatment ensures enhanced efficacy of said further medicament. In addition, the use of a binding molecule of the invention in such combination treatment ensures returned responsiveness to said further medicament when compared to individuals not suffering form the disease.

It was shown that inhibition of the pathway involving stimulation of monocytes/macrophages and T-cells via a co-stimulatory molecule on said cells results in more differentiated cells in the mentioned cultures or the circulation. Either one of the co-stimulatory molecules is thus a target in the treatment of immune cell mediated bone marrow failure. Preferred examples of co-stimulatory molecules are the B7 antigens, the CD40 receptor and the CD40 receptor ligand (CD154). However, several other co-stimulatory molecules exist, such as the tumor necrosis factor receptor (TNFR) family members CD27, CD30, CD137 (4-1BB), HVEM, GITR and OX40 (CD134) (ref: Watts T H, Annu Rev Immunol 2005(23)23-68). CD40 may function as a master switch for T cell co-stimulation because of its ability to induce B7 family ligands as well as several TNF family ligands on dendritic cells (DCs) (refs 1, 13-18 from Watts T H).

In a particularly preferred embodiment the co-stimulatory molecule is the CD40-receptor on monocytes/macrophages or the CD40 ligand (CD154) on lymphocytes, particularly T-cells. In a preferred embodiment said T-cell is a stimulatory T-cell. Preferably said stimulatory T-cell is a T-helper cell, preferably at CD4⁺ T-cell.

The binding molecule can be any type of binding molecule that comprises specificity for the co-stimulatory molecule. In a preferred embodiment said binding molecule is a protein or proteinaceous binding molecule. Preferably, said binding molecule belongs to any family of non-antibody scaffold protein binders such as, but not limited to, anticalins, C-type lectin domain binders, avimers, Adnectins, and DARPins (Designed Ankyrin Repeat Proteins) (ref. Sheridan C. Nature Biotechnology 2007, (25), 365-366.)

Preferably said binding molecule at least comprises a variable domain of a heavy chain or a light chain of an antibody or an equivalent thereof. Non-limiting examples of such proteins are VHH, nanobodies, Human Domain Antibodies (dAbs), Unibody, Shark Antigen Reactive Proteins (ShArps), Small Modular ImmunoPharmaceutical (SMIP™) Drugs, monobodies and/or IMabs (ref. Sheridan C. Nature Biotechnology 2007, (25), 365-366.). Preferred are binding molecules that have at least a variable domain of a heavy chain and a light chain of an antibody or equivalents thereof. Non-limiting examples of such binding molecules are F(ab)-fragments and Single chain Fv fragments. Many different proteins exist that have an IG-fold that can be manipulated to specifically bind a target. Such manipulated proteins are considered equivalents of an antibody. In a preferred embodiment said binding molecule comprises an antibody. The antibody may be a natural antibody or a synthetic antibody. In a preferred embodiment an antibody comprises the CDR1, CDR2, CDR3 regions of an antibody. However, artificial generation of CDR like regions such as can be selected for instance via phage display are also included in the present invention. In a preferred embodiment said antibody is a human, humanized or human-like antibody. Particularly preferred are binding molecules that (apart from their specificity) do not further interact with the immune system. In case of antibodies it is preferred that said antibody comprises an IgG4 constant region, or an IgG4 like constant region. Preferably said constant region is a human constant region. For instance it is possible to mutate the constant region of an IgG1 molecule such that it no longer activates the complement system upon binding to its target.

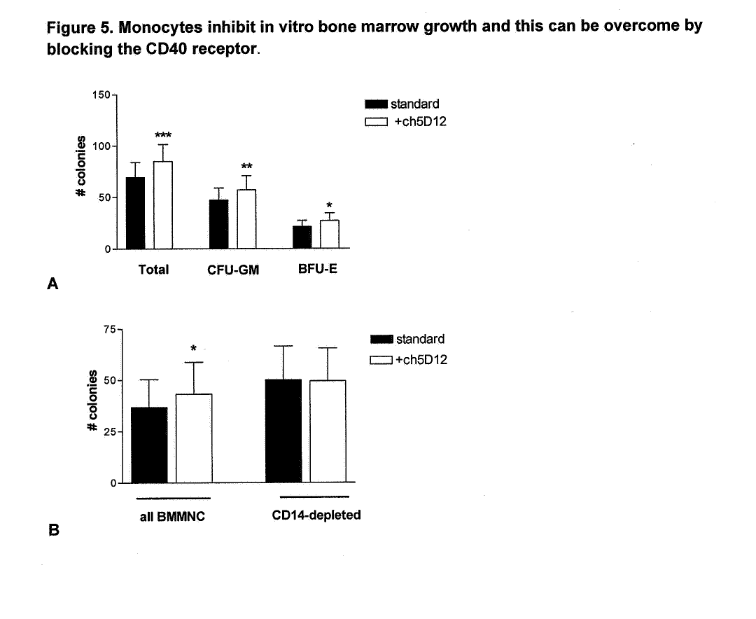

Said binding molecule is preferably a non-stimulatory and/or antagonistic binding molecule. Such molecules bind to the co-stimulatory molecule they are specific for and prevent and/or inhibit signalling (activation) through the co-stimulatory molecule. In a preferred embodiment said binding molecule is a CD40 specific antibody or equivalent thereof. Preferably said antibody is a non-stimulatory CD40 specific antibody. Most preferably said antibody is the murine anti-human CD40 monoclonal antibody 5D12, the chimeric 5D12 antibody or a deimmunized 5D12 antibody. The variable regions of the light and heavy chain of murine 5D12 and deimmunized 5D12 are depicted in FIG. 5 (FIG. 5). In another embodiment said antibody is the fully human anti-CD40 monoclonal antibody 15B8 (WO2002028904).

In another embodiment said binding molecule is a CD40L (CD154) specific antibody or equivalent thereof. Preferably said antibody is monoclonal antibody 5c8 having ATCC Accession No. HB-10916 or monoclonal antibody IDEC131 (Dumont F J, Curr Opin Investig Drugs. 2002 May; 3(5):725-34). The invention further provides use of a binding molecule specific for a co-stimulatory molecule for stimulating the production of differentiated haematopoietic cells in a culture comprising haematopoietic progenitor cells, lymphocytes (particularly T-cells) and monocytes/macrophages/dendritic cells. Further provided is the use of a binding molecule specific for a co-stimulatory molecule for the preparation of a medicament for the treatment of immune cell mediated bone marrow failure. In yet another embodiment the invention provides the use of a binding molecule specific for a co-stimulatory molecule for stimulating the production of differentiated haematopoietic cells in a culture comprising haematopoietic progenitor cells, lymphocytes (particularly T-cells) and monocytes/macrophages/dendritic cells.

Further provided is the use of a binding molecule specific for a co-stimulatory molecule for the preparation of a medicament for the treatment of immune cell mediated bone marrow failure.

### EXAMPLES

### Patients, Materials and Methods

Patient data and samples. All sample prelevations and handling were conducted in accordance with the guidelines of the local ethical committee of the University Hospital of Leuven, which comply with the Helsinki declaration. Samples from 95 different patients with histologically proven MDS were used for this study. Patient characteristics are summarized in table 1. The samples were obtained at routine peripheral blood (PB) and BM sampling after informed consent. The majority of patients (70 of 95 patients) did not receive therapy for MDS at the time the samples were collected. Data on cytogenetic analysis were provided by the Center for Human Genetics of the University Hospital of Leuven and the Cytogenetic Lab of the Bordet Institute of Brussels.

In addition, PB samples were obtained from 60 healthy volunteers. Donors (n=29) used in the flow cytometry studies were age-matched (mean age 68, range 59-82) with the MDS patients. BM aspirates of 11 healthy young volunteers (mean age 21, range 19-24) were also obtained to serve as control samples.

Hematologic values were determined with a Sysmex XE-2100 automated Hematology Analyzer. Serum C-reactive protein (CRP) concentration was measured on an Hitachi Modular Analytics D automated analyzer with a particle-enhanced immunoturbidimetric method (CRPLX, Roche Diagnostics).

Flow cytometry. PB and BM samples were collected in sodium EDTA coated tubes and were processed immediately. After lysis with 10:1 NH₄Cl buffer and washing with phosphate buffered saline (PBS, Bio-Whittaker Europe, Cambrex, Belgium), cells were immunostained with the following fluorescein isothiocyanate (FITC), phycoerythrin (PE) and peridinin chlorophyll (PerCP) conjugated monoclonal antibodies (MoAbs): anti-CD3, anti-CD4, anti-CD14, anti-CD40L, anti-CD40, anti-CD80, anti-CD86, anti-CD45 (all from Becton Dickinson, Erembodegem, Belgium). Appropriate isotype-matched negative controls were used for each sample. Cells were incubated with 10 μl of the monoclonal antibody for 30 minutes at 4° C. in the dark, subsequently washed once and resuspended in 400 μl CellFIX (Becton Dickinson). Samples were analyzed on a FACScan or FACSCanto Flowcytometer (Becton Dickinson). To determine the phenotype of monocytes, we gated on the CD14+ population with intermediate side scatter (SSC) and enumerated the percentage of cells in this population that was double-positive for CD14 and CD40, CD14 and CD80, and CD14 and CD86. Similarly, we gated on the population with low SSC that was CD4 and CD3 double-positive, to determine the percentage of this population that expressed CD40L.

Monocyte isolation and stimulation. PB mononuclear cells (PBMNCs) were isolated over a Ficoll-Hypaque (Lymphoprep, Nycomed Pharma, Oslo, Norway) step gradient and washed once in PBS and subsequently in MACS buffer. CD14+ cells were isolated by MACS using CD14+ magnetic beads (Miltenyi Biotec, Bergisch Gladbach, Germany) according to the manufacturer's instructions. Isolated CD14+ cells were enumerated using trypan blue exclusion. They were subsequently plated in Iscove's modified Dulbecco's Medium (IMDM, Gibco BRL Life Technologies, Paisley, UK) supplemented with penicillin and streptomycin (Gibco BRL Life Technologies, Paisley, UK) and 15% fetal bovine serum (FBS, Stem Cell Technologies, Vancouver, BC, Canada) in wells of 24-well plates (Falcon Multiwell, Becton Dickinson) at a density of 3×10⁵ cells per mL per well. After 1 week of culture in a humidified incubator at 37° C. with 5% CO₂, the medium was replaced with fresh medium supplemented with either lipopolysaccharide (LPS, Santa Cruz Biotechnology, Germany) at 1 μg/mL or a mixture of clone 64 (agonist anti-human CD40 monoclonal antibody, PanGenetics, The Netherlands) at 10 μg/mL, and interferon-gamma (IFN-γ, Santa Cruz Biotechnology, Germany) at a final concentration of 1000 IU/mL. Thalidomide (Sigma, Germany) dissolved in DMSO at a stock-concentration of 5 μg/μL was added in various concentrations (5-10-25 μg/ml) immediately thereafter. After 24 h, supernatant was harvested and stored at −20° C. until further analysis.

Colony forming assay. Following the enumeration of viable cells, BMMNC (5×10⁵ for MDS patients, 2.5×10⁵ for controls) were prepared in MethoCult H4434 media containing 30% FBS, 50 ng/mL stem cell factor, 10 ng/mL granulocyte-macrophage colony stimulating factor, 10 ng/mL interleukin-3, and 3 U/mL erythropoietin (StemCell Technologies, Vancouver, BC, Canada). From each sample, a tube without and a tube with ch5D12 (antagonist chimeric anti-human CD40 antibody, PanGenetics, The Netherlands) at a final concentration of 10 μg/mL was prepared and plated in duplicate wells. Following 14 days of incubation, plates were scored using an inverted microscope. On the basis of cell number and colony morphology, we defined granulocyte-monocyte colony forming units (CFU-GM), and erythroid colony forming units, either single or clustered, as BFU-E.

Enzyme-linked immunosorbent assay. Commercially available ELISA-kits for detection of TNF-α (BD, Pharmingen), IL-16 (R&D systems), IL-6 (PeproTech Ltd, UK) and IL-10 (PeproTech Ltd, UK) were used per mannufacturer's recommendations. The sensitivity limit of the ELISAs was 1.25, 4, 16 and 20 pg/mL, respectively.

Statistics. All statistical analyses were performed using Prism 3.0 software. We used the Mann-Whitney u-test to compare means between two groups. To analyze the results of the BM cultures and the cytokine concentrations in presence/absence of thalidomide, we used Wilcoxon matched pairs tests. The Spearman's test was used to analyze covariance between 2 variables. P-values represent double-sided tests. P-values<0.05 were considered significant.

### Results

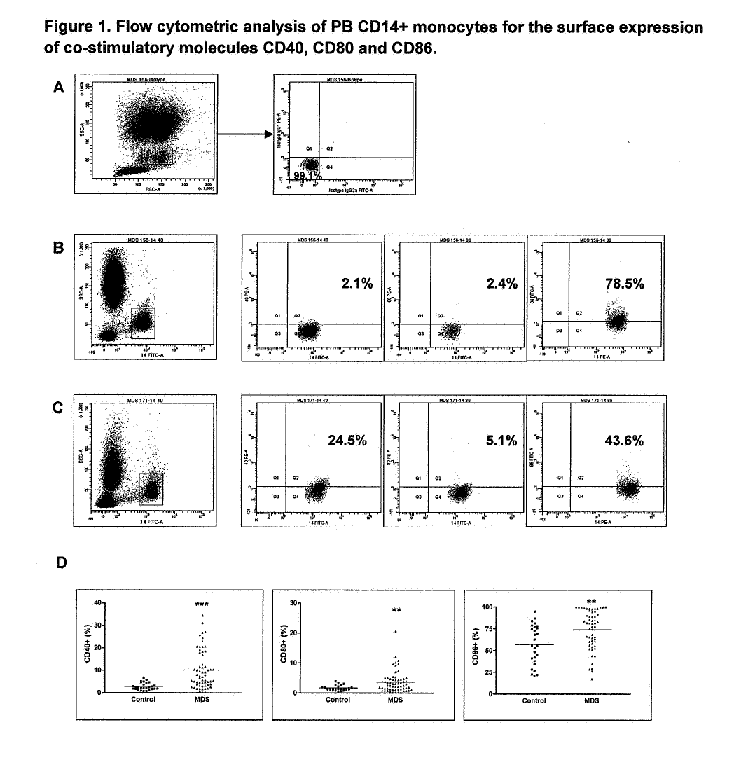

Increased expression levels of CD40, CD80 and CD86 on MDS monocytes. Using flow cytometry, we analysed the phenotype of circulating monocytes in the PB of 61 MDS patients. According to the FAB classification 37 patients were diagnosed with refractory anemia (RA), 20 with refractory anemia with ringed sideroblasts (RARS) and 4 with refractory anemia with excess blasts (RAEB); and according to the WHO classification 1 patient was diagnosed with RA, 30 with refractory cytopenia with multilineage dysplasia (RCMD), 1 with RARS, 19 with refractory cytopenia with multilineage dysplasia and ringed sideroblasts (RCMD-RS), 3 with a 5q-syndrome, 3 with RAEB-1, 1 with RAEB-2, and 3 with unclassified MDS (MDS-u). The majority did not receive any treatment for MDS prior to sample collection (40 of 61). Therapies for MDS included transfusion (n=9), growth-factors (4 rhEPO, 1 rhG-CSF and 1 combination of rhEPO and rhG-CSF), iron-chelation therapy alone (n=2) or a combination of these therapies (n=4). To exclude any influence of age on the phenotype of monocytes, PB of 29 age-matched subjects served as control group. We defined monocytes as those cells with intermediate SSC characteristics that expressed CD14. We gated on this population to enumerate the percentage of monocytes co-expressing CD40 (see FIG. 1B). Upon activation through T cells expressing CD40L, monocytes up-regulate expression of CD40 but also of the B7-molecules CD80 and CD86^{18,19}. We therefore also analysed expression levels of CD80 and CD86.

Absolute numbers of circulating monocytes were comparable in both groups. MDS patients had significantly more circulating CD40+(10.2%+/−1.1 vs. 2.8%+/−0.3, p<0.0001), CD80+(3.8%+/−0.5 vs. 1.7%+/−0.2, p=0.004) and CD86+(74.1%+/−3.0 vs. 56.9%+/−4.3, p=0.001) monocytes compared to age-matched control subjects. The same observation was made in the BM of 23 patients (data not shown). We found a highly significant correlation between the percentage of CD40+ and CD80+ monocytes (Spearman r=0.54, p<0.0001), indicating that both activation markers are expressed at high levels in the same patients. Expression of CD86, however, did not correlate significantly with either CD40 or CD80 expression levels, presumably because even in the absence of significant CD40 or CD80 expression, monocytes already express significant levels of CD86.

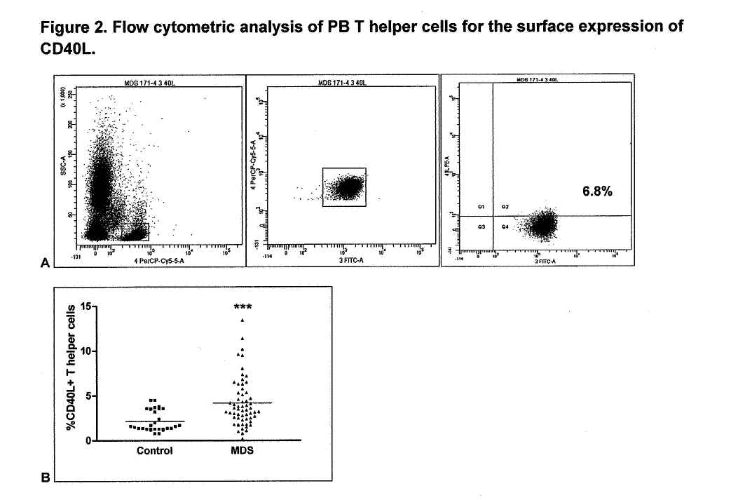

Increased expression of CD40L on MDS T helper cells. We also evaluated expression of CD40L (CD154) on the surface of T helper cells in the same group of patients and normal controls. T helper cells were defined as being CD3+/CD4+ cells with low SSC characteristics. CD40L was found to be significantly more expressed on T helper cells from MDS patients (4.2%+/−0.4 vs. 2.2%+/−0.2, p<0.0001, see FIG. 2) compared to age-matched controls. This was also found for T helper cells present in BM aspirates of MDS patients (5.0%+/−0.8 vs. 2.6%+/−0.5, p<0.01).

Furthermore, we found a correlation between expression of CD40 by monocytes and CD40L expression by T helper cells (Spearman r=0.32, p=0.02), indicating that activation of monocytes and lymphocytes occurs in the same patients.

Activation of the CD40 receptor induces higher cytokine production in MDS. We next evaluated if TNF-α production differs when monocytes from MDS patients or healthy control donors are stimulated with an agonist anti-human CD40 antibody or LPS. We therefore isolated CD14+ cells from PB of 20 MDS patients and 25 healthy controls. According to the FAB-classification, these MDS patients were diagnosed as RA (n=12), RARS (n=6), RAEB (n=2), and as RCMD (n=10), RCMD-RS (n=6), RAEB-1 (n=2), 5q-syndrome (n=1) and MDS-u (n=1) according to the WHO classification. After 7 days of culture to induce maturation, CD14+ cells were subjected for 24 hours to two different stimuli to induce TNF-α production: LPS or an agonist anti-human CD40 mAb (clone 64, PanGenetics, The Netherlands²⁶). In order to maximize the response to the given stimulus, we used a high concentration of LPS (1 μg/mL). For the same reason, we combined the agonist anti-CD40 antibody with IFN-γ (final concentration 1000 IU/mL), as IFN-γ is known to induce transcription of the CD40-receptor²⁷.

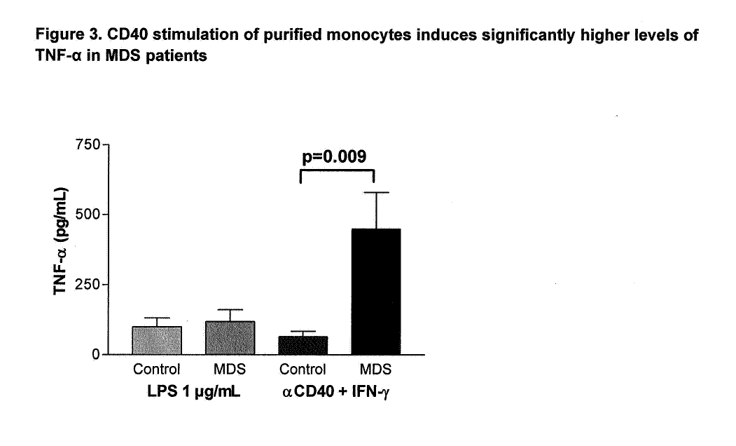

The mean purity of CD14+ isolation was 97.1% (range 91.9-99.7%) for controls and 97.2% (range 95.0-98.9%) for MDS patients. TNF-α levels of unstimulated CD14+ cells were low (3+/−1 pg/mL) in both groups. As shown in FIG. 3, LPS induced similar levels of TNF-α in patients (123+/−44 pg/mL) and controls (100+/−32 pg/mL). In contrast, the agonist anti-CD40 antibody combined with IFN-γ induced a significantly higher TNF-α production by CD14+ monocytes from MDS patients compared to normal controls (449+/−131 vs. 67+/−20 pg/mL, p=0.009).

In a subsequent study, we determined also the level of other cytokines produced by monocytes from both healthy and MDS donors. As summarized in table 3, we repeatedly found that monocytes from MDS patients produced significantly more TNF-α in response to clone 64, whilst producing comparable TNF-α levels in response to LPS. In control monocytes, the presence of clone 64 and IFN-γ did not lead to a higher IL-18 production than in absence of a stimulus, whereas in MDS monocytes it did (p=0.003). Spontaneous IL-6 production was lower in MDS monocytes (p=0.0009) as was the IL-6 production induced by LPS (p=0.006). Whilst in controls the IL-6 production after CD40 stimulation was not significantly different from the condition without a stimulus, in patients this stimulus led to a significantly higher IL-6 production (p=0.0003). Finally, in patients, CD40 stimulation led to significantly higher IL-10 concentrations than stimulation with LPS, unlike in healthy controls (p=0.0002).

### Effect of Thalidomide on Stimulated Monocytes

The therapeutic effect of thalidomide in patients with MDS has been ascribed to its property to inhibit TNF-α production. Having shown that TNF-α production, but also IL-16, IL-6 and IL-10 production, is up-regulated in monocytes from patients with MDS in response to CD40-activation, we next studied the effect of thalidomide on LPS-induced and CD40-induced cytokine production in both monocytes from controls and monocytes from patients with MDS.

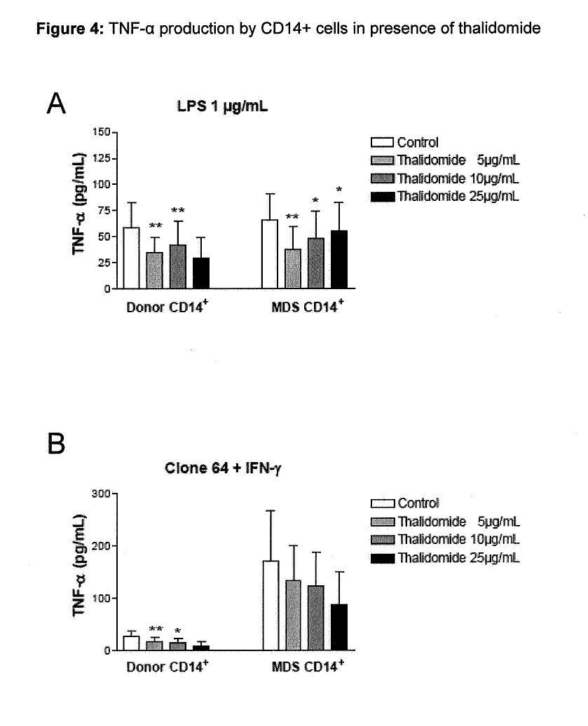

In control CD14+ cells thalidomide significantly inhibited LPS-induced TNF-α production (FIG. 4A). The concentrations of IL-1β, IL-6 or IL-10 were not significantly influenced by the presence of thalidomide (in any concentration), and are therefore not shown. Also in MDS-derived CD14+ cells, TNF-α production after stimulation with LPS was significantly lower with every concentration of thalidomide used (FIG. 4A). IL-16 and IL-6 concentrations induced by LPS-stimulation were higher than in presence of thalidomide, unlike in donors. IL-10 concentrations were not affected by the presence of thalidomide (results not shown).

TNF-α production of control monocytes stimulated with clone 64 was significantly down-regulated in a dose-dependent way by the presence of thalidomide, as shown in FIG. 4B. IL-16 and IL-6 production were significantly higher in the presence of thalidomide (data not shown). IL-10 concentration was unaltered by the presence of thalidomide. In monocytes from MDS patients however, TNF-α production was decreased in the presence of 25 μg/mL thalidomide, as is shown in FIG. 4B. IL-16, IL-6 and IL-10 concentrations were not significantly different in the presence of thalidomide. In conclusion, thalidomide inhibits LPS-induced TNF-α production in both monocytes from controls and monocytes from patients with MDS. But whereas thalidomide also inhibits TNF-α production in control monocytes stimulated by CD40-agonists, higher concentrations of thalidomide were able to inhibit TNF-α production from MDS-derived monocytes.

Correlation between expression of CD40, CD80 and CD86 by monocytes and clinical characteristics. We evaluated whether there was a correlation between the expression of CD40 and CD40L, on CD14+ monocytes and T helper lymphocytes respectively, and disease or patient characteristics. First, we correlated the percentage of CD40+ or CD80+ monocytes with hematological values: hemoglobin levels, platelet, white blood cell and absolute neutrophil count (ANC). We observed a negative correlation between ANC and the percentage of circulating CD80+ monocytes (r=−0.26, p<0.05). The same observation was made for marrow samples where the percentage of CD80+ monocytes inversely correlated with PB ANC at the time of BM sampling (r=−0.51, p<0.05). There was no significant correlation between expression of CD40 or CD86 and hematological parameters, Of note, in healthy donors we also found an inverse correlation between ANC and the percentage of CD80+ monocytes (r=−0.49, p<0.01). Although this type of analysis does not allow demonstration of a causal relationship, this suggests that activation of monocytes can be related to lower ANC values, irrespective of disease status. We found no correlation between blast-percentage and the expression of co-stimulatory molecules. We also correlated expression of CD40, CD80 and CD86 on monocytes and CD40L on T helper cells with serum concentrations of CRP. CRP levels were generally low and independent of expression of co-stimulatory molecules. Clinical signs or a history of auto-immunity were observed in 5 patients, but did not relate to the level of CD40, CD80 or CD86 expression. Subsequently, we determined if expression of CD40, CD80, or CD86 on monocytes correlated with disease status according to the FAB subtype, WHO subtype, IPSS score, cytogenetics, gender or previous treatment. We found no differences between RA, RARS and RAEB patients. Interestingly, we found that patients with the karyotypic abnormality trisomy 8 had the highest level of circulating CD40+ monocytes. Patients with intermediate-1 IPSS score had significantly more CD40+ monocytes than low-risk patients, but this was attributable to the fact that all trisomy 8 patients had an intermediate-1 risk score. There were no differences between patients that had previously received treatment and treatment-naïve patients with regards to the expression of CD40 or CD80 by monocytes.

Finally, we separated patients into 2 groups based on the percentage CD40 expressing monocytes, using the highest value of CD40 expressing monocytes found in the control group as the cut-off value (6.4%): one group of patients with <6.4% CD40+ monocytes, and a group of patients with >6.4% CD40+ monocytes. High levels of CD40+ monocytes coincided with a higher IPSS score, intermediate and poor cytogenetic risk score, age below 60 y and presence of trisomy 8 (see table 2). The latter 2 factors have been identified in multivariate analyses as being independent factors predictive of response to immunosuppressive therapy. Of note, patients with favourable karyotypic abnormalities (3 patients with del(5q), 2 patients with del(20q) and 2 men with −Y) had low expression of CD40 on their circulating monocytes.

Antagonizing the CD40 receptor on monocytes leads to increased colony formation in MDS. As we demonstrated that the CD40 receptor was up-regulated on monocytes and that activation of this receptor leads to increased TNF-α production, we hypothesized that interference with CD40 signaling may affect haematopoietic progenitor growth in vitro. To test this hypothesis, we used an antagonist anti-CD40 mAb (ch5D12, PanGenetics, The Netherlands) to inhibit CD40 signaling²². Ch5D12 is a chimeric antibody, having the variable region (both Vh and Vl) of the antibody 5D12m of FIG. 5 fused to the constant region of a human antibody.

We plated BMMNCs of 26 patients in the presence or absence of ch5D12. Patients were diagnosed as RA (n=17), RARS (n=4) and RAEB (n=5) according to FAB, and as RCMD (n=14), 5q-(n=2), MDS-u (n=1), RCMD-RS (n=4), RAEB-1 (n=2) and RAEB-2 (n=3) according to WHO. Significant more CFU-GM and BFU-E grew in the presence of ch5D12 compared with cultures without ch5D12 (see FIG. 5A). To prove that this effect was monocyte-dependent, we also added ch5D12 to cultures of BMMNC depleted of CD14 positive cells by MACS columns. The presence of ch5D12 did not alter colony formation of CD14-depleted BMMNC, whereas ch5D12 increased colony production in the same samples in presence of all BMMNC (see FIG. 5B). We did not observe this effect of ch5D12 on CFU-GM and BFU-E colony number in BMMNC cultures of healthy controls (n=9).

To further substantiate that CD40-CD40L interaction may affect haematopoietic progenitors we examined whether a correlation exists between the number of CD40L+ T helper cells or CD40+ monocytes within BMMNC. We demonstrated an inverse correlation between the number of BFU-E and the percentage of CD40L+ T helper cells in that particular patient group (r=−0.54, p=0.03). Although there was also a trend for lower numbers of CFU-GM with increasing percentage of CD40+ monocytes, this was not significant (r=−0.42, p=0.07).

### Discussion

Cytopenia is the presenting symptom of most patients with MDS and is the leading cause of morbidity and mortality. The presence of abnormal clonal progenitor cells plays a pivotal role in the development of marrow failure, and immune enhancing mechanisms contribute to a variable extent to this process. Most previous work has focused on the role of lymphocytes in this process. Autologous T cells inhibit growth of progenitor cells in MDS^{2,3}, and this effect can disappear after successful treatment with ATG². A role of immune mediated suppression of haematopoiesis has been most extensively defined in trisomy 8 MDS. Consistent with what was shown for the general population of MDS patients^{4,6,7}, Sloand and co-workers have demonstrated that expanded clonal populations of T cells can be found in all patients with trisomy 8⁵. In addition, they showed that the suppressive action of autologous T cells in these patients is restricted to CD8+ cells with this specific clonotype.

Notwithstanding these insights, it is still unknown how immune mediated mechanisms contribute to the pancytopenia seen in MDS. In trisomy 8 patients responding to immunosuppressive therapy, the number of progenitor cells with an extra chromosome 8 increases⁵, suggesting that the expanded clonal population of T lymphocytes in these patients controls the growth of genetically abnormal haematopoietic progenitors. Marrow failure associated with this immune response that appears to be directed specifically at the malignant clone, must therefore come from bystander effects on remaining normal haematopoietic cells. It is not known which factors cause this bystander phenomenon, and several mechanisms have been suggested for this inhibition of normal progenitor cells by T cells⁵: release of cytokines by activated T cells²⁸ or cross-recognition of targets through molecular mimicry or epitope spreading²⁹.

The aim of this study was to investigate the possible role of monocytes in this immune-mediated process. Monocytes are the main source of TNF-α. Activated T cells can induce TNF-α production by monocytes via CD40-CD40L interactions. The CD40 receptor is a 45-50 kDa type I phosphoprotein that is a member of the TNF receptor (TNFR) superfamily. It has been demonstrated that monocytes express low levels of CD40³⁰, but expression of the CD40 gene is induced upon activation, which is most pronounced by stimulation with IFN-γ²⁷. Besides production of TNF-α, CD40 ligation also results in the secretion of other pro-inflammatory cytokines and chemokines such as IL-1, IL-6, IL-8, IL-10, IL-12 and macrophage-inflammatory protein-10^{8,19}. In addition, ligation of CD40 by CD40L, present on T helper cells, promotes up-regulation of co-stimulatory molecules (CD40, CD80, CD86, MHC class II molecules) and FasL. CD40L is transiently expressed on activated T helper cells and has been shown to be present on PB T helper cells in several auto-immune conditions.

Since lymphocytes in MDS have been found to have an activated phenotype^{7,31}, this pathway could be responsible for activation of monocytes in BM of MDS patients, for production of cytokines including TNF-α and subsequent suppression of haematopoiesis.

In the present invention we demonstrate a role of CD40-CD40L interactions in the pathogenesis of MDS-related marrow failure. First of all, we have shown in a large group of 61 predominantly lower-risk untreated patients, that significantly more PB CD14+ monocytes express CD40 and CD80. In addition, significantly more PB T helper cells express CD40L in MDS patients, and expression of CD40L on T lymphocytes was highly correlated with CD40 expression of CD14+ monocytes. Over-expression of CD40 and CD40L was also observed in BM samples, where lymphocytes and monocytes are thought to exert their function.

Besides measurement of expression levels of CD40 and CD40L, we also analyzed the in vitro effects of stimulation and inhibition of this pathway on cytokine production, including TNF-α production, and proliferation and differentiation of haematopoietic progenitors. Ligation of CD40 on MDS monocytes using the agonist anti-CD40 mAb resulted in increased TNF-α production compared to controls. Furthermore, it was demonstrated that MDS monocytes also produce relatively more IL-18, IL-6 and IL-10 than wild-type monocytes as a result of CD40 stimulation. To the best of our knowledge, this is the first report demonstrating different behavior of MDS monocytes with regards to TNF-α production. These results underline the importance of the CD40-CD40L pathway in MDS. In our series, TNF-α production was similar between MDS and normal monocytes stimulated with LPS, consistent with previous reports by others¹⁷. Increased surface expression of the CD40 receptor on MDS monocytes could account for this different behavior, but also activation of different downstream pathways might be responsible³⁰, but this remains to be explored.

TNF-α has been shown to inhibit hematopoiesis and has been implicated in the pathogenesis of MDS-related BM failure (Gersuk G M. et al., Br. J. Haematol. 1998(103)176-188). Thalidomide and other immune-modulatory drugs are believed to have a wide mode of action including inhibition of TNF-α production by monocytes. The inhibitory action of thalidomide on TNF-α production has been ascribed to enhancing degradation of TNF-α-mRNA (Moreira A L et al., J. Exp. Med. 1993(177)1675-1680) meaning that it is not specifically targeting LPS downstream signaling pathways. Activation of monocytes through CD40 leads to activation of MAPK pathways and eventually to NF-kB activation (Rothe M et al., Science 1995(269)1424-1427; Van Kooten C., et al., J. Leukocyte Biol. 2000(67)2-17)). We have therefore analyzed thalidomide's activity on CD40-induced TNF-α production and to date, we have been the first to report this. We demonstrate that thalidomide is capable of inhibiting TNF-α production induced by CD40 stimulation of monocytes from healthy controls. However, in patients, higher doses of thalidomide were needed to achieve this effect. This observation is in line with the observation that relatively high doses are needed to achieve responses in MDS. These doses are however hard to reach due to dose-limiting toxicity, especially neurologic side effects. We have shown that a binding molecule specific for a co-stimulatory molecule expressed on monocytes/macrophages and/or dendritic cells can at least in part return responsiveness to these monocytes/macrophages and/or dendritic cells for a further medicament for the treatment of MDS. We further show that a binding molecule specific for a co-stimulatory molecule expressed on monocytes/macrophages and/or dendritic cells increases responsiveness of said further medicament for the treatment of MDS. Without being bound by theory, it is believed that reducing the immune-stimulatory potential of the monocytes/macrophages and/or dendritic cells is beneficial for the further MDS treatment.

In addition, we analyzed the effects of inhibiting this pathway in colony formation assays, using the monoclonal antibody ch5D12, which is an antagonist chimeric anti-human CD40 antibody that has been shown to be effective in the therapy of inflammatory bowel disease²² and non-human primate models of autoimmune encephalomyelitis²⁴. In the presence of ch5D12, the number of BFU-E and CFU-GM significantly increased in cultures of MDS BMMNC, but not following depletion of CD14+ monocytes prior to initiating the colony forming assay. This proves that the inhibitory effects of monocytes on haematopoiesis can be overcome by antagonizing the CD40-receptor on these monocytes using ch5D12. Together with the observation that the number of BFU-E inversely correlates with the percentage of CD40L+ T helper cells, this suggests that the inhibitory effect of lymphocytes on haematopoiesis can be partially attributed to additional cytokine-release from monocytes. However, it is still unclear which cytokines are responsible for this. Because the TNF-α concentrations in our BM cultures were very low, we were unable to observe down-regulation of TNF-α levels in presence of ch5D12 (data not shown).

Taken together, these observations have both diagnostic as therapeutic value. First, we have shown that expression of CD40 by MDS monocytes serves as a marker for an on-going immune attack inside the BM. Although CD40 expression on circulating monocytes is increased in patients with severe sepsis³², a systemic inflammatory response is not likely to be responsible for our results. CRP levels were low and did not correlate with expression of CD40 on CD14+ cells. In addition, patients with a recent history of infection were excluded from this study. The use of CD40 as a marker of immunological activity in the marrow is additionally supported by the observation that age below 60 y and trisomy 8 coincide with high CD40 expression. Both factors have been identified as the strongest predictors for response to immunosuppressive therapy in MDS^{14,33} and therefore immune mechanisms play a particular important role in these patients.

Inhibition of CD40 signaling on monocytes results in increased BFU-E and CFU-GM formation. Furthermore, the number of CFU-GM inversely correlated with the percentage of CD40+ CD14+ cells, even though this did not reach statistical significance. In addition, increased percentages of CD14+ monocytes that co-express co-stimulatory molecules coincided with decreased ANC, an observation made in the 61 blood samples. Moreover, this observation was independently made in 23 BM samples, that were obtained from different patients. The last observation was also made in PB from the age-matched controls, therefore suggesting that it is not specific for MDS, but is a general phenomenon in all immune cell mediated bone marrow failures.

Previous studies have demonstrated that CD8+ T cells have an activated phenotype in MDS patients^{7,31}. Here we show that also CD4+ T cells are activated in patients and demonstrate their involvement in MDS pathogenesis through CD40-CD40L interactions. This is further supported by the observation that CD40L expression by T helper cells coincided with CD40 expression by monocytes. Furthermore, the number of BFU-E in MDS BMMNC cultures significantly decreased with increasing CD40L expression by T helper cells. The high percentage of circulating CD40L-expressing T helper cells is intriguing. It indicates that CD40L expression is either increased or stabilized in MDS. The increased CD40L expression may be involved through antigen presenting cells in the induction of the suppressive CD8 clonotype, but this also warrants further investigation.

Apart from their diagnostic value, our observations also have therapeutic relevance as antagonizing the CD40-CD40L interaction can be used to treat MDS-related cytopenia. Antagonist CD40 antibodies increases blood counts, reduce anemia symptoms, and/or reduce dependence on transfusions. Monoclonal antibodies against CD40 are currently under investigation for use in various haematological disease, such as multiple myeloma^{34,35} and non-Hodgkin's lymphoma³⁶. The mode of action of these antibodies relies on antibody-dependent cell-mediated cytotoxicity³⁷ and by a direct apoptotic effect on malignant cells³⁴. In other malignancies, activation of monocytes with agonist anti-CD40 Mab has been shown to inhibit tumor growth³⁸. In MDS however, the therapeutic value lies in the tapering the immune response and hence limiting collateral damage on normal progenitors in order to improve blood counts. For this reason, inhibition rather than stimulation of the CD40-CD40L pathways is therapeutically relevant in MDS. The antagonist anti-CD40 ch5D12 described here is relevant option to target inhibition of CD40-CD40L in human pathologies including MDS.

The data demonstrating higher levels of TNF-α production by CD40-stimulated MDS monocytes when compared to control monocytes and LPS-stimulated monocytes, results in higher doses thalidomide to dampen these high TNF-α levels. Indeed, this observation is in line with the observation that relatively high doses thalidomide are used to achieve responses in MDS. Therefore, the inhibition of a co-stimulatory pathway, preferably the CD40-CD40L pathway, is not only be an attractive therapy for tapering the immune response in MDS as a stand alone therapy, but can also be used in combination of other therapies to further potentiate the overall therapeutic effect. The inhibition of the CD40-CD40L pathway permits less frequent dosing and lower doses of currently used (investigational) drugs, including thalidomide, for the treatment of MDS, thereby decreasing the chance for unwanted side effects.

### Tables

TABLE 1
Clinical characteristics of 95 MDS patients

| | Number |
|---|---|
| Gender (M/F) | 56/39 |
| Mean age (range), years | 71 (32-90) |
| FAB diagnosis | |
| RA | 60 |
| RARS | 25 |
| RAEB | 10 |
| WHO diagnosis | |
| RA | 2 |
| RCMD | 50 |
| RARS | 1 |
| RCMD-RS | 24 |
| 5q- | 4 |
| RAEB-1 | 6 |
| RAEB-2 | 4 |
| MDS-u | 4 |
| Cytogenetics | |
| Normal | 41 |
| Abnormal | 37 |
| Favorable | 12 |
| Intermediate | 13 |
| Poor | 12 |
| N.A.¹ | 17 |
| IPSS | |
| Low | 38 |
| Intermediate-1 | 30 |
| Intermediate-2 | 8 |
| High | 2 |
| MDS treatment | |
| None | 70 |
| Transfusions | 12 |
| rhEPO/rhG-CSF/both | 7/1/2 |
| Iron-chelation | 4 |

MDS, myelodysplastic syndromes;
FAB, French-American-British;
RA, refractory anemia;
RARS, refractory anemia with ringed-sideroblasts;
RAEB: refractory anemia with excess blasts;
WHO: World Health Organization;
RCMD, refractory cytopenia with multilineage dysplasia;
RCMD-RS, refractory cytopenia with multilineage dysplasia and ringed-sideroblasts;
MDS-u, MDS-unclassified;
IPSS, International Prognostic Scoring System
¹not available

TABLE 2
Comparative analysis of laboratory and clinical findings between
patients with high and low levels of circulating CD40+ monocytes.

| | CD40 high (n = 33) | CD40 low (n = 28) | p-value |
|---|---|---|---|
| CD40+ (% of monocytes) | 16.0 +/− 1.3 | 3.3 +/− 0.3 | p < 0.0001^{a} |
| CD80+ (% of monocytes) | 5.1 +/− 0.7 | 2.2 +/− 0.3 | p = 0.001^{a} |
| CD86+ (% of monocytes) | 77.4 +/− 3.6 | 70.2 +/− 4.9 | n.s.^{a} |
| Age (years) | 68 +/− 2 | 74 +/− 2 | n.s.^{a} |
| Hemoglobin (g/dL) | 10.1 +/− 0.4 | 10.0 +/− 0.4 | n.s.^{a} |
| Platelets (×10³/μL) | 185 +/− 32 | 190 +/− 34 | n.s.^{a} |
| White blood cell count (/μL) | 4662 +/− 357 | 5389 +/− 631 | n.s.^{a} |
| Absolute neutrophils count (/μL) | 2657 +/− 327 | 2619 +/− 333 | n.s.^{a} |
| Gender (male/female) | 19/14 | 18/10 | n.s.^{b} |
| Age <60 y | 9 of 33 | 1 of 28 | p = 0.01^{b} |
| FAB (RA/RARS/RAEB) | 18/12/3 | 19/8/1 | n.s.^{b} |
| WHO (RA/RCMD/RCMD-RS/RARS/ | 0/17/11/1 | 1/13/8/0 | n.s.^{b} |
| 5q-/RAEB-1/RAEB-2/MDS-u) | 0/2/1/1 | 3/1/0/2 | |
| IPSS (low/int-1/int-2/high)¹ | 7/13/3/1 | 17/7/1/0 | p = 0.02^{b} |
| Anemia (Hb < 10 g/dL) | 19 of 33 | 16 of 28 | n.s.^{b} |
| Thrombocytopenia (<100 × 10³/μL) | 12 of 33 | 9 of 28 | n.s.^{b} |
| Neutropenia (<1500/μL) | 8 of 33 | 9 of 28 | n.s.^{b} |
| Abnormal karyotype¹ | 13 of 24 | 10 of 25 | n.s.^{b} |
| Favorable/Intermediate/Poor² | 0/8/5 | 7/2/1 | p = 0.001^{b} |
| Trisomy 8¹ | 4 of 24 | 0 of 25 | p = 0.03^{b} |
| MDS-treatment (yes/no)³ | 8 of 33 | 13 of 28 | n.s.^{b} |
| Auto-immune disease⁴ | 3 of 33 | 2 of 28 | n.s.^{b} |

Patients are grouped into a “high” and “low” group, using the highest measured value in the control group as cut-off point (6.4%). B/Table summarizes results of (a) Mann-Whitney u-tests to compare means in both groups, numbers are represented as mean +/− SEM; and (b) X²-tests for categorical variables.
¹From 49 of the 61 patients, cytogenetic data were available to determine IPSS score.
²IPSS defines the karyotypic abnormalities-Y, del(20q) and del(5q) as favorable; trisomy 8, single miscellaneous and double miscellaneous abnormalities as intermediate; and 3 or more abnormalities and any chromosome 7 abnormality as poor.
³MDS-treatment included growth-factors (4 rhEPO, 1 rhG-CSF, 1 with a combination of rhEPO and rhG-CSF), transfusions (n = 9), iron chelation-therapy (n = 2), or a combination (n = 4).
⁴Auto-immune disease included vasculitis (n = 1), rheumatoid arthritis (n = 1), auto-immune hemolytic anemia (n = 1), rheumatoid arthritis combined with ITP (n = 1), inflammatory bowel disease (n = 1).

TABLE 3
Cytokine production by purified CD14+ cells

| | Donor | MDS | P-value |
|---|---|---|---|
| TNF-α (pg/mL) | | | |
| Un-stimulated | 3 ± 1 | 3 ± 1 | n.s. |
| LPS | 80 ± 24 | 90 ± 28 | n.s. |
| Clone 64 + IFN-γ | 52 ± 15 | 293 ± 89 | 0.03 |
| IL-1β (pg/mL) | | | |
| Un-stimulated | 10 ± 2 | 6 ± 2 | n.s. |
| LPS | 19 ± 5 | 12 ± 2 | n.s. |
| Clone 64 + IFN-γ | 13 ± 2 | 18 ± 7* | n.s. |
| IL-6 (pg/mL) | | | |
| Un-stimulated | 1693 ± 514 | 657 ± 274 | 0.0009 |
| LPS | 4190 ± 1370 | 1618 ± 356 | 0.006 |
| Clone 64 + IFN-γ | 1486 ± 284 | 1207 ± 363** | n.s. |
| IL-10 (pg/mL) | | | |
| Un-stimulated | 516 ± 169 | 239 ± 76 | 0.047 |
| LPS | 519 ± 123 | 371 ± 102 | n.s. |
| Clone 64 + IFN-γ | 1169 ± 291 | 1646 ± 393*** | n.s. |

CD14+ cells were purified from PB using MACS columns. After 7 days in culture, CD14+ cells were stimulated with fresh medium supplemented with either LPS (1 μg/mL) or a mixture of clone 64 (10 μg/mL) and IFN-γ (1000 IU/mL). A control condition received only fresh medium (“un-stimulated”). After 24 h supernatant was collected. P-values represent two-sided Mann-Whitney u-tests to compare means of MDS patients and controls. Stimulation of MDS monocytes with clone 64 and IFN-γ also resulted in a significant increase in the production of IL-1β, IL-6 and IL-10 compared to the “un-stimulated” condtion (p-values were 0.003*, 0.0003** and 0.0002***, respectively, Wilcoxon matched pairs test), unlike in donor monocytes.

### Legends to FIGS. 1 to 6

FIG. 1. Flow cytometric analysis of PB CD14+ monocytes for the surface expression of co-stimulatory molecules CD40, CD80 and CD86. Total PB cells were lysed, washed and subsequently double stained with PE- or FITC-conjugated mouse antihuman anti-CD14, anti-CD40, anti-CD80 and anti-CD86 mAb. (A) Representative dot plot of the setting of quadrants. Cells were stained with appropriate isotype control antibodies, and analysis was performed in the monocyte-gate. (B-C) Representative dot plot of determination of percentage of monocytes that expressed CD40, CD80 and CD86. Analysis was performed on CD14+ cells with intermediate SSC. (B) represents results of a 57 y female patient with RARS/RCMD-RS with del(20)(q12) with low level of expression of co-stimulatory molecules; (C) represents results of a 78 y female patient with RA/RCMD and normal cytogenetics with high expression of co-stimulatory molecules; (D) Results of PB samples of 61 patients and 29 age-matched controls. Patient distribution according to FAB: RA (n=37), RARS (n=20), RAEB (n=4); according to WHO RA (n=1), RCMD (n=30), RCMD-RS (n=19), RARS (n=1), 5q-(n=3), RAEB-1 (n=3), RAEB-2 (n=1), MDS-u (n=3). Plots show results of Mann-Whitney u-tests to compare means of controls and patients. P-values were <0.0001***; 0.004** and 0.001** respectively.

FIG. 2. Flow cytometric analysis of PB T helper cells for the surface expression of CD40L. PB were lysed, washed and subsequently triple stained with CD40L-PE, CD3-FITC and CD4-PerCp. T helper cells were defined as CD3+/CD4+ double positive cells with low SSC characteristics. (A) Representative dot plot demonstrating a patient with increased proportion of activated T helper cells. Quadrants were determined on basis of appropriate isotype control antibodies. (B) In total 29 age-matched controls and 60 patients were available for comparison for mean percentage of CD40L+ T helper cells (p<0.0001, Mann-Whitney u-test).

FIG. 3. CD40 stimulation of purified monocytes induces significantly higher levels of TNF-α in MDS patients. CD14+ cells were isolated from PB of 25 healthy controls and 20 patients with MDS. After 7 days of culture, these cells were stimulated with LPS (1 μg/mL) or clone 64 (10 μg/mL, activating anti-CD40 mAb) and IFN-gamma (1000 IU/mL). Supernatant was collected after 24 h and TNF-α concentration was measured with ELISA. P-value represents result of Wilcoxon matched pairs test.

FIG. 4. TNF-α production by CD14+ cells in the presence of thalidomide. After 7 days in culture, purified CD14+ cells were stimulated with fresh medium supplemented with either LPS (1 μg/mL) (A) or a mixture of clone 64 (10 μg/mL) (B) and IFN-γ (1000 IU/mL), in the presence of increasing doses of thalidomide. After 24 h, supernatant was harvested for subsequent determination of TNF-α concentration. P-values represent Wilcoxon matched pairs test between the conditions with and without thalidomide (*p<0.05, **p<0.001).

FIG. 5. Monocytes inhibit in vitro bone marrow growth and this can be overcome by blocking the CD40 receptor. BMMNCs were grown in methylcellulose supplemented with growth factors (M4434, StemCell technologies) in standard conditions or in the presence of ch5D12 (antagonist chimeric anti-CD40 mAb). After 14 days, CFU-GM and BFU-E were scored. Results are shown as number of colonies per 250×10³ plated BMMNCs. (A) BMMNCs of 26 patients were cultured in M4434 in presence/absence of ch5D12. Patients were diagnosed according to FAB as RA (n=17), RARS (n=4), RAEB (n=5); and according to WHO as RCMD (n=14), 5q-(n=2), MDS-u (n=1), RCMD-RS (n=4), RAEB-1 (n=2) and RAEB-2 (n=3). The presence of the ch5D12 significantly increased colony formation. P-values represent results of Wilcoxon matched pair tests and were 0.0007***, 0.005** and 0.02* respectively. (B) Of 10 patients BMMNCs and BMMNCs depleted of CD14+ cells were cultured in presence/absence of ch5D12. The significant increase (p<0.05*) in total number of colonies was not observed in CD14-depleted conditions.

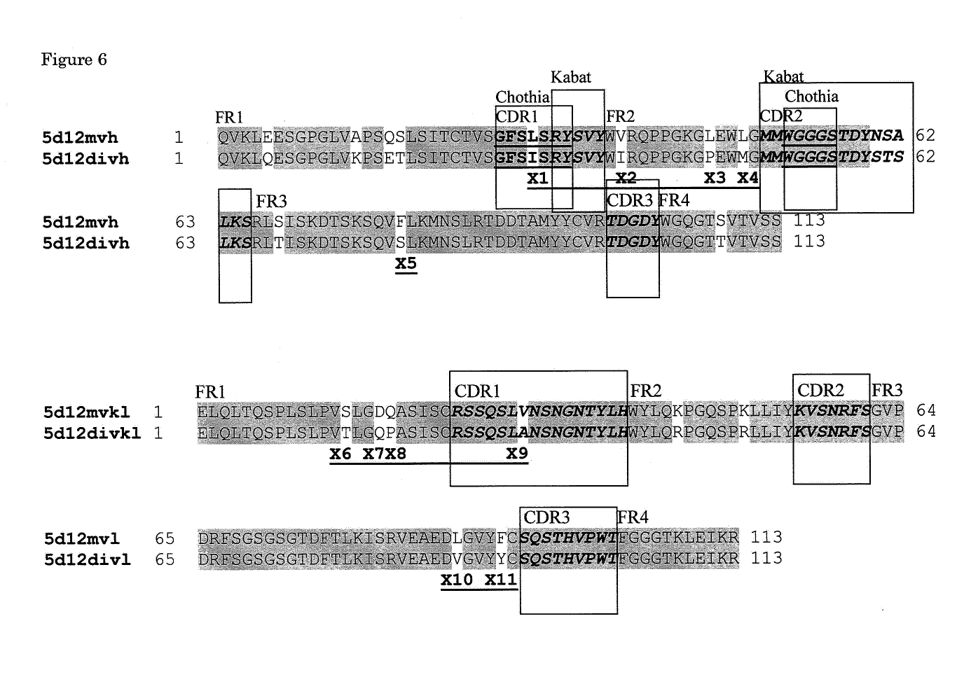

FIG. 6. 5D12m: Murine anti human CD40 receptor antibody. 5D12di: Variant of heavy and light chain of 5D12m. Vh=Variable heavy chain, V1=variable light chain. FR 1-4 (Framework 1-4); Chothia; KABAT position of CDR regions.

### REFERENCES

1. Delforge M. Understanding the pathogenesis of myelodysplastic syndromes. Hematol J. 2003; 4:303-309.
2. Molldrem J J, Jiang Y Z, Stetler-Stevenson M, Mavroudis D, Hensel N, Barrett A J. Haematological response of patients with myelodysplastic syndrome to antithymocyte globulin is associated with a loss of lymphocyte-mediated inhibition of CFU-GM and alterations in T-cell receptor Vbeta profiles. Br J Haematol. 1998; 102:1314-1322.
3. Baumann I, Scheid C, Koref M S, Swindell R, Stern P, Testa N G. Autologous lymphocytes inhibit hemopoiesis in long-term cultures in patients with myelodysplastic syndromes. Exp Hematol. 2002; 30:1405-1411.
4. Epperson D E, Nakamura R, Saunthararajah Y, Melenhorst J J, Barrett A J. Oligoclonal T cell expansion in myelodysplastic syndrome: evidence for an autoimmune process. Leuk Res. 2001; 25:1075-1083.
5. Sloand E M, Mainwaring L, Fuhrer M, et al. Preferential suppression of trisomy 8 compared with normal haematopoietic cell growth by autologous lymphocytes in patients with trisomy 8 myelodysplastic syndrome. Blood. 2005; 106:841-851.
6. Wlodarski M W, Gondek L P, Nearman Z P, et al. Molecular strategies for detection and quantitation of clonal cytotoxic T-cell responses in aplastic anemia and myelodysplastic syndrome. Blood. 2006; 108:2632-2641.
7. Epling-Burnette P K, Painter J S, Rollison D E, et al. Prevalence and clinical association of clonal T-cell expansions in Myelodysplastic Syndrome. Leukemia. 2007; 21:659-667.
8. Jonasova A, Neuwirtova R, Cermak J. Cyclosporin A therapy in hypoplastic MDS patients and certain refractory anaemias without hypoplastic bone marrow. Br J Haematol. 1998; 200:304-309.
9. Tichelli A, Gratwohl A, Wuersch A, Nissen C, Speck B. Antilymphocyte globulin formyelodysplastic syndrome? Br J Haematol. 1988; 68:139-140.
10. Biesma D, van den Tweel J, Verdonck L. Immunosuppressive therapy for hypoplastic myelodysplastic syndrome. Cancer. 1997; 79:1548-1551.
11. Molldrem J, Caples M, Mavroudis D, Plante M, Young N S, Barrett A J. Antithymocyte globulin (ATG) abrogates cytopenias in patients with myelodysplastic syndrome. Br J Haematol. 1997; 99:699-705.
12. Molldrem J, Rivera M, Bahceci E. Treatment of bone marrow failure of myelodysplastic syndrome with antithymocyte globulin. Ann Intern Med. 2002; 137:156-163.
13. Broliden P A, Dahl I M, Hast R, et al. Antithymocyte globulin and cyclosporine A as combination therapy for low-risk non-sideroblastic myelodysplastic syndromes. Haematologica. 2006; 91:667-670.
14. Barrett J, Sloand E, Young N. Determining which patients with myelodysplastic syndrome will respond to immunosuppressive treatment. Haematologica. 2006; 91:583-584.
15. Kitagawa M, Saito I, Kuwata T, et al. Overexpression of tumor necrosis factor (TNF)-alpha and interferon (IFN)-gamma by bone marrow cells from patients with myelodysplastic syndromes. Leukemia. 1997; 11:2049-2054.
16. Parker J, Mufti G. The myelodysplastic syndromes: a matter of life or death. Acta Haematologica. 2004; 111:78-99.
17. Visani G, Zauli G, Tosi P, et al. Impairment of GM-CSF production in myelodysplastic syndromes. Br J Haematol. 1993; 84:227-231.
18. Grewal I S, Flavell R A. CD40 and CD154 in cell-mediated immunity. Annu Rev Immunol. 1998; 16:111-135.
19. van Kooten C, Banchereau J. CD40-CD40 ligand. J Leukoc Biol. 2000; 67:2-17.
20. Harigai M, Hara M, Nakazawa S, et al. Ligation of CD40 induced tumor necrosis factor-alpha in rheumatoid arthritis: a novel mechanism of activation of synoviocytes. J Rheumatol. 1999; 26:1035-1043.
21. Sekine C, Yagita H, Miyasaka N, Okumura K. Expression and function of CD40 in rheumatoid arthritis synovium. J Rheumatol. 1998; 25:1048-1053.
22. Kasran A, Boon L, Wortel C H, et al. Safety and tolerability of antagonist anti-human CD40 Mab ch5D12 in patients with moderate to severe Crohn's disease. Aliment Pharmacol Ther. 2005; 22:111-122.
23. Carlsen H S, Yamanaka T, Scott H, Rugtveit J, Brandtzaeg P. The proportion of CD40+ mucosal macrophages is increased in inflammatory bowel disease whereas CD40 ligand (CD154)+ T cells are relatively decreased, suggesting differential modulation of these costimulatory molecules in human gut lamina propria. Inflamm Bowel Dis. 2006; 12:1013-1024.
24. t Hart B A, Blezer E L, Brok H P, et al. Treatment with chimeric anti-human CD40 antibody suppresses MRI-detectable inflammation and enlargement of pre-existing brain lesions in common marmosets affected by MOG-induced EAE. J Neuroimmunol. 2005; 163:31-39.
25. Carayanniotis G, Masters S R, Noelle R J. Suppression of murine thyroiditis via blockade of the CD40-CD40L interaction. Immunology. 1997; 90:421-426.

26. Companjen A R, van der Wel L I, Boon L, Prens E P, Laman J D. CD40 ligation-induced cytokine production in human skin explants is partly mediated via IL-1. Int Immunol. 2002; 14:669-676.
27. Benveniste E N, Nguyen V T, Wesemann D R. Molecular regulation of CD40 gene expression in macrophages and microglia. Brain Behav Immun. 2004; 18:7-12.
28. Maciejewski J, Selleri C, Anderson S, Young N S. Fas antigen expression on CD34+ human marrow cells is induced by interferon gamma and tumor necrosis factor alpha and potentiates cytokine-mediated haematopoietic suppression in vitro. Blood. 1995; 85:3183-3190.
29. Eisen H N. Specificity and degeneracy in antigen recognition: yin and yang in the immune system. Annu Rev Immunol. 2001; 19:1-21.
30. Burger D, Molnarfi N, Gruaz L, Dayer J M. Differential induction of IL-1beta and TNF by CD40 ligand or cellular contact with stimulated T cells depends on the maturation stage of human monocytes. J Immunol. 2004; 173:1292-1297.
31. Kook H, Zeng W, Guibin C, Kirby M, Young N S, Maciejewski J P. Increased cytotoxic T cells with effector phenotype in aplastic anemia and myelodysplasia. Exp Hematol. 2001; 29:1270-1277.
32. Sugimoto K, Galle C, Preiser J C, Creteur J, Vincent J L, Pradier O. Monocyte CD40 expression in severe sepsis. Shock. 2003; 19:24-27.
33. Saunthararajah Y, Nakamura R, Wesley R, Wang Q J, Barrett A J. A simple method to predict response to immunosuppressive therapy in patients with myelodysplastic syndrome. Blood. 2003; 102:3025-3027.
34. Tai Y T, Catley L P, Mitsiades C S, et al. Mechanisms by which SGN-40, a humanized anti-CD40 antibody, induces cytotoxicity in human multiple myeloma cells: clinical implications. Cancer Res. 2004; 64:2846-2852.
35. Tai Y-T, Li X-F, Catley L, et al. Immunomodulatory Drug Lenalidomide (CC-5013, IMiD3) Augments Anti-CD40 SGN-40-Induced Cytotoxicity in Human Multiple Myeloma: Clinical Implications. Cancer Res. 2005; 65:11712-11720.
36. Fanale M A, Younes A. Monoclonal Antibodies in the Treatment of Non-Hodgkin's Lymphoma. Drugs. 2007; 67:333-350.
37. Hayashi T, Treon S P, Hideshima T, et al. Recombinant humanized anti-CD40 monoclonal antibody triggers autologous antibody-dependent cell-mediated cytotoxicity against multiple myeloma cells. Br J Haematol. 2003; 121:592-596.
38. Lum H D, Buhtoiarov I N, Schmidt B E, et al. Tumoristatic effects of anti-CD40 mAb-activated macrophages involve nitric oxide and tumour necrosis factor-alpha. Immunology. 2006; 118:261-270.

1. A method for culturing a collection of cells comprising haematopoietic progenitor cells, lymphocytes (preferably T-cells) and monocytes/macrophages and/or dendritic cells comprising culturing said cells or precursors thereof in the presence of a binding molecule specific for a co-stimulatory molecule expressed on said monocytes/macrophages and/or dendritic cells or said lymphocytes (preferably T-cells).

2. A method for stimulating the production of differentiated haematopoietic cells in a culture comprising haematopoietic progenitor cells, lymphocytes (preferably T-cells) and monocytes/macrophages and/or dendritic cells comprising culturing said cells or precursors thereof in the presence of a binding molecule specific for a co-stimulatory molecule expressed on said monocytes/macrophages and/or dendritic cells or said lymphocytes (preferably T-cells).

3. A method according to claim 1, wherein said monocytes/macrophages and/or dendritic cells are derived from an individual suffering from or at risk of suffering from immune cell mediated bone marrow failure.

4. A method according to claim 3, wherein said immune cell mediated bone marrow failure comprises a clonal bone marrow disorder.

5. A method according to claim 3, wherein said clonal bone marrow disorder is a myelodysplastic syndrome (MDS), aplastic anemia, paroxysmal nocturnal hemoglobinuria (PNH), pure red cell aplasia (PRCA), myelofibrosis or large granular lymphocytic leukemia (LGL).

6. A method for the treatment of cytopenia and/or bone marrow failure in an individual comprising providing said individual with a binding molecule specific for a co-stimulatory molecule expressed on monocytes/macrophages and/or dendritic cells or lymphocytes (preferably T-cells).

7. A method for the treatment of cytopenia and/or bone marrow failure in an individual comprising providing said individual with a binding molecule for reducing and/or inhibiting immune-cell activation in said individual.

8. A method according to claim 1, wherein said co-stimulatory molecule comprises CD40 or CD40L (CD154).

9. A method according to claim 1, wherein said lymphocyte is a T-cell, preferably a stimulatory T-cell.

10. A method according to claim 1, wherein said binding molecule comprises an antibody.

11. A method according to claim 1, wherein said antibody is a human, humanized or human-like antibody.

12. A method according to claim 1, wherein said binding molecule is a non-stimulatory and/or antagonistic binding molecule.

13. A method according to claim 1, wherein said binding molecule comprises a CD40 specific antibody.

14. A method according to claim 13, wherein said antibody is a non-stimulatory CD40 specific antibody.

15. A method according to claim 1, wherein said haematopoietic progenitor cells are of the myeloid lineage.

16. A method according to claim 15, wherein said haematopoietic progenitor cells produce differentiated granulocytes and/or monocytes/macrophages and/or dendritic cells.

17. A method according to claim 1, further comprising the administration of a further medicament for the treatment of MDS.

18. A method according to claim 1, further comprising the administration of an immune-modulatory drug, chemotherapy, or an antibody.

19. A method according to claim 18, wherein said immune-modulatory drug is thalidomide, or an analogue.

20. A method according to claim 19, wherein said analogue is lenalomide.

21. A method according to claim 18, wherein said antibody is an anti-TNF-α antibody.

22. Use of a binding molecule specific for a co-stimulatory molecule for stimulating the production of differentiated haematopoietic cells in a culture comprising haematopoietic progenitor cells, lymphocytes (particularly T-cells) and monocytes/macrophages and/or dendritic cells.

23. Use of a binding molecule specific for a co-stimulatory molecule for the preparation of a medicament for the treatment of immune cell mediated bone marrow failure.


SEQUENCE LISTING (4 sequences)

SEQ ID NO: 1 - protein, 113 aa, Mus musculus
QVKLEESGPGLVAPSQSLSITCTVSGFSLSRYSVYWVRQPPGKGLEWLGMMWGGGSTDYNSALKSRLSISKDTSKSQVFLKMNSLRTDDTAMYYCVRTDGDYWGQGTSVTVSS

SEQ ID NO: 2 - protein, 113 aa, Mus musculus
QVKLQESGPGLVKPSETLSITCTVSGFSISRYSVYWIRQPPGKGPEWMGMMWGGGSTDYSTSLKSRLTISKDTSKSQVSLKMNSLRTDDTAMYYCVRTDGDYWGQGTTVTVSS

SEQ ID NO: 3 - protein, 113 aa, Mus musculus
ELQLTQSPLSLPVSLGDQASISCRSSQSLVNSNGNTYLHWYLQKPGQSPKLLIYKVSNRFSGVPDRFSGSGSGTDFTLKISRVEAEDLGVYFCSQSTHVPWTFGGGTKLEIKR

SEQ ID NO: 4 - protein, 113 aa, Mus musculus
ELQLTQSPLSLPVTLGQPASISCRSSQSLANSNGNTYLHWYLQRPGQSPRLLIYKVSNRFSGVPDRFSGSGSGTDFTLKISRVEAEDVGVYYCSQSTHVPWTFGGGTKLEIKR